(19) United States
(12) Patent Application Publication
(10) Pub. No.: US 2005/0265944 A1
(43) Pub. Date: Dec. 1, 2005

(54) Carbohydrate-based anti-wrinkle and tissue remodelling compounds

(75) Inventor: William Butler Cowden, Kambah (AU)

Correspondence Address: NIXON PEABODY, LLP, 401 9TH STREET, NW, SUITE 900, WASHINGTON, DC, 20004-2128, US

(73) Assignee: Ultraceuticals R&D Limited, New South Wales (AU)

(21) Appl. No.: 10/504,562

(22) PCT Filed: Feb. 17, 2003
(86) PCT No.: PCT/AU03/00199

§ 371 Date: Jun. 30, 2005

(30) Foreign Application Priority Data
Feb. 15, 2002 (AU) PS 0528

(51) Int. Cl. A61K 7/06; A61K 31/715

(52) U.S. Cl. 424/70.13; 514/54

(57) ABSTRACT

A process and cosmetic preparation for skin augmentation of a subject comprising an active component comprising a 1,4 linked D-glucose oligosaccharide or polysaccharide wherein after delivery, the oligosaccaharide or polysaccharide causes an accumulation of fibroblasts in the skin at or near to the site of delivery and induces production of collagen in the skin.

### TECHNICAL FIELD

The present invention relates to carbohydrates and carbohydrate-containing compounds that possess anti-wrinkle and tissue remodelling activity in the skin. In particular, the invention relates to the use of these compounds as anti-wrinkle and tissue remodelling agents in animals and humans.

### BACKGROUND

The cutaneous tissue contains cellular protein and glycoprotein components which together influence the thickness and form of the tissue. Fibroblasts are a common cellular constituent of the skin and produce various proteins that are important structural components of cutaneous tissue. One such protein is collagen which can be and is widely used to artificially augment cutaneous shape (Klein, A W et al, 1997). A characteristic of ageing and wrinkled skin is a reduction in cellularity (Gilchrest and Chiu, 1995; Fenske and Lober, 1986; Contet-Audonneau et al, 1999). A potential goal of treating ageing or wrinkled skin could be to increase the cellularity in the affected area and a further desirable effect of this increase in cellularity would be the increased production of extracellular dermal components including collagen. One of the clinical features of treating damaged skin with retinoic acids, believed to be of cosmetic benefit, is an increase in new collagen synthesis (Gilchrest, 1997). Another commonly used treatment for skin wrinkling, glycolic acid, has been shown to increase collagen synthesis in fibroblast cultures in vitro (Moy et al., 1996) and it has been suggested that this effect may occur in vivo and account for the apparent beneficial effects associated with glycolic acid use. It has also been proposed that a deficiency of superficial dermal collagen is the main cause of photo-ageing (Kang et al., 1997).

U.S. Pat. No. 5,980,916 entitled, “Use of laminarin and oligosaccharides derived therefrom in cosmetics and for preparing a skin treatment drug” by Yvin et al, describes the use of laminarin or laminarin-derived oligosaccharides as a “cosmetic or pharmaceutical, particularly dermatological”. Laminarin is a polysaccharide derived from Laminaria spp seaweed and is a linear polymer composed of beta-1,3-D-glucose and a small amount of beta-1,6-D-glucose linkages. This patent notes that laminarin, oligosaccharides derived therefrom, and compositions containing these substances have “stimulating, regenerating, conditioning and energising effects on human dermis fibroblasts and human epidermis keratinocytes”. It does not teach the use of any oligosaccharides or polysaccharide other than that from laminarin as a process for “stimulating skin cells selected from the group consisting of fibroblasts and keratinocytes comprising delivering to said skin cells an active component consisting essentially of laminarin in an amount effective for stimulating said skin cells”. This patent clearly teaches that laminarin and oligosaccharides derived therefrom have a “stimulating effect” on skin cells in culture. It does not teach that skin cells in vivo are similarly stimulated. It does not teach that laminarin and oligosaccharides derived therefrom produce augmentation of skin defects. It also does not teach that laminarin and oligosaccharides derived therefrom have any activity when applied to the skin of a living mammal. It does not teach or imply that another oligosaccharides or polysaccharide could have skin augmenting or tissue remodelling activity.

U.S. Pat. No. 5,916,880 entitled, “Reduction of skin wrinkling using sulphated sugars” by Bar-Shalom, et al, describes the use of “a sulphated saccharide or a salt or complex thereof for the preparation of a medicament for topical application to the skin”. This patent claims a method for “cosmetically treating skin to reduce wrinkles, the method comprising topically applying to affected skin areas a cosmetically effective amount of at least one compound selected from the group consisting of sulfated monosaccharides, sulfated disaccharides, and salts and complexes thereof”. This patent does not claim the use of any other than sulfated sugars for the treatment of skin wrinkles. The patentee states that the saccharide is preferably a polysulphated or persulphated saccharide, which means that two or more, possibly all, sulphur-containing moieties are present as substituents on the carbohydrate moiety. The patent claims the possible use of the sulfated mono and disaccharides with non-sulfated oligosaccharides or poly saccharides but no enabling disclosures are made. The compounds and methods disclosed in this patent do not teach or imply that non-sulfated oligosaccharides or polysaccharides could have a skin augmenting or tissue remodelling activity.

The present inventor has found that a number of carbohydrates (oligosaccharides and polysaccharides) have skin augmenting or tissue remodelling activity.

### SUMMARY OF INVENTION

The present invention generally provides use of agents which, when applied to or into skin, can attract fibroblasts and stimulate production of collagen to provide augmentation of the treated skin.

In a first aspect, the present invention provides a process for skin augmentation of a subject, the process comprising delivering to the skin an active component comprising a 1,4 linked D-glucose oligosaccharide or polysaccharide wherein after delivery, the oligosaccharide or polysaccharide causes an accumulation of fibroblasts in the skin at or near to the site of delivery and induces production of collagen in the skin.

Preferably, the oligosaccharide or polysaccharide is selected from the group consisting of D-mannose polysaccharide (mannan) from Saccharomyces cerevisiae; exocellular phosphomannan produced by Pichia holstii, purified D-mannose high molecular weight acid-resistant polysaccharide core of the exocellular phosphomannan produced by Pichia holstii; 6-O-phospho-alpha-D-mannose-(1,3)alpha-D-mannose-(1,3) alpha-D-mannose-(1,3)alpha-D-mannose-(1,2)-alpha-D-mannose, beta-1,4-D-mannose oligosaccharides isolated by acid hydrolysis of the mannan isolated from the seeds of Phoenix canariensis; beta-1,4-mannopentaose, beta-1,4-mannohexaose; beta-1,4-mannoheptaose, beta-1,4-mannooctaose, beta-1,4-mannononaose, beta-1,4-mannodecanose, beta-1,4-mannoundecanose, beta-1,4-mannododecanose, amylopectin, amylose, 1,4-D-glucose oligosaccharides isolated by acid hydrolysis of amylose, maltopentaose, maltohexaose, maltoheptaose, alpha-1,4-maltooctaose, maltononaose, maltodecanose, maltoundecanose; maltododecanose, mixtures or combinations thereof, physically modified analogues thereof, and chemically modified analogues thereof.

In one preferred form, the oligosaccharide or polysaccharide is maltopentaose.

In another preferred from, the oligosaccharide or polysaccharide is 1,4 beta-manohexaose.

In another preferred from, the oligosaccharide or polysaccharide is O-phosphorylated mannan.

In another preferred from, the oligosaccharide or polysaccharide is amylose or amylopectin.

In another preferred from, the oligosaccharide or polysaccharide is purified D-mannose high molecular weight acid-resistant polysaccharide fragment (polysaccharide core fraction) of the exocellular phosphomannan produced by Pichia holstii. Preferably, the purified D-mannose high molecular weight acid-resistant polysaccharide fragment comprises 6-O-phospho-alpha-D-mannose-(1,3)-alpha-D-mannose-(1,3)alpha-D-mannose-(1,3)-alpha-D-mannose-(1,2)-alpha-D-mannose.

The present invention also includes the use of chemically modified analogues of the oligosaccharides or polysaccharides, such modification enhancing transdermal penetration or solubility. For example, a lipid moiety, such as palmitic add, attached to oligosaccharide or polysaccharide, might enhance transdermal penetration. An insoluble carbohydrate, such as amylose, might be chemically modified to enhance aqueous solubility and enable its use in an injectable formulation.

Preferably, the subject is a human requiring wrinkle reduction or skin augmentation.

The oligosaccharide or polysaccharide can be injected directly into the skin or applied topically to the skin.

The oligosaccharide or polysaccharide can delivered to the skin at a concentration of about 0.001 to 100% by weight. Preferably, the oligosaccharide or polysaccharide is delivered to the skin at a concentration of about 0.01 to 70% by weight. More preferably, the oligosaccharide or polysaccharide is delivered at a concentration of about 0.1 to 30% by weight. Even more preferably, the oligosaccharide or polysaccharide is delivered at a concentration of about 1 to 20% by weight.

In a second aspect, the present invention provides use of a 1,4 linked D-glucose oligosaccharide or polysaccharide for skin augmentation of a subject wherein after delivery of the oligosaccharide or polysaccharide to the skin, fibroblasts are caused to accumulate in the skin at or near to the site of delivery and collagen is caused to be produced in the skin.

In a third aspect, the present invention provides a cosmetic preparation for skin augmentation of a subject comprising an effective amount of a 1,4 linked D-glucose oligosaccharide or polysaccharide in a suitable diluent or excipient, the oligosaccharide or polysaccharide being capable of causing an accumulation of fibroblasts in the skin and inducing production of collagen in the skin.

Preferably, the oligosaccharide or polysaccharide is selected from the group consisting of D-mannose polysaccharide (mannan) from Saccharomyces cerevisiae; exocellular phosphomannan produced by Pichia holst1i, purified D-mannose high molecular weight acid-resistant polysaccharide core of the exocellular phosphomannan produced by Pichia holstii; 6-O-phospho-alpha-D-mannose-(1,3)-alpha-D-mannose-(1,3)-alpha-D-mannose-(1,3)-alpha-D-mannose-(1,2)-alpha-D-mannose, beta-1,4-D-mannose oligosaccharides isolated by acid hydrolysis of the mannan isolated from the seeds of Phoenix canariensis; beta-1,4-mannopentaose, beta-1,4-mannohexaose; beta-1,4-mannoheptaose, beta-1,4-mannooctaose, beta-1,4-mannononaose, beta-1,4-mannodecanose, beta-1,4-mannoundecanose, beta-1,4-mannododecanose, amylopectin, amylose, 1,4-D-glucose oligosaccharides isolated by acid hydrolysis of amylose, maltopentaose, maltohexaose, maltoheptaose, alpha-1,4-maltooctaose, maltononaose, maltodecanose, maltoundecanose; maltododecanose, and mixtures or combinations thereof.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 shows histological sections of rat skin tissue from Example 6 showing an increase in cellularity 1 week following intradermal injection of O-phosphomannan.

FIG. 2 shows a histological section of rat skin tissue from Example 7 showing an influx of fibroblastoid cells (immunohistochemical staining with murine anti-rat prolyl 4-hydroxylase monoclonal antibody, counterstained with hemotoxylin and eosin) 1 week following injection with amylopectin.

FIG. 3 shows Gomori's (collagen) stained histological sections of rat skin tissue from Example 7 injected one week earlier with either (A) amylopectin or (B) saline control (50 microliters).

### MODE(S) FOR CARRYING OUT THE INVENTION

It has now been discovered and is the subject of the present invention that an increase in cellularity in cutaneous tissue can be induced by applying certain carbohydrates to skin tissue. The increase in cellularity appears to be brought about through migration of cells into the area exposed to the carbohydrates. It has also been found that the arrival of these new cells is correlated with an increase in collagen deposition within the area treated with these carbohydrates. It has been discovered that not all carbohydrates have this cell attracting activity and thus activity in this regard is structure-dependent. It is believed by the present inventors that the action of these carbohydrates may be due to specific interactions with cells rather than a non-specific action. It has further been discovered that the newly arrived cells in the treated dermis are largely fibroblastic cells.

Many of the carbohydrates of the present invention that are active in promoting fibroblast migration are naturally occurring oligosaccharides and polysaccharides or derivatives thereof. Some oligosaccharides and polysaccharides that are based on glucose have been found to increase the cellularity and collagen deposition in cutaneous tissue. Such oligosaccharides and polysaccharides include the 1,4-alpha-D-glucose oligosaccharides, which are commonly referred to as maltose oligosaccharides. These include maltose oligosaccharides that contain from 4 to 12 glucose units including maltotetraose, maltopentaose, maltohexaose, maltoheptaose, maltooctaose, maltononaose, maltodecanose, maltoundecanose and maltododecanose and mixtures of maltose oligosaccharides containing varying amounts of these oligosaccharides. The maltose polysaccharide amylopectin is particularly effective in this regard and various hydrolysates of starch containing the above mentioned oligosaccharides, also have this property.

As noted above, some oligosaccharides and polysaccharides based upon glucose, such as maltose oligosaccharides have skin augmenting activity. It was discovered in work leading to the present invention that not all glucose-based oligosaccharides and polysaccharides are equally potent in causing an increase in cellularity and collagen deposition in treated skin. Thus, the oligosaccharides and polysaccharides consisting of 1,6-alpha-D-glucose linkages, dextran oligosaccharides and dextran polysaccharides (dextrans), are much less potent than the 1,4-alpha-D-glucose linked maltose oligosaccharides and polysaccharides. The efficacy of the 1,6-alpha-D-glucose oligosaccharides and polysaccharides is so low that from a practical standpoint these agents would be considered by a clinical practitioner to be ineffective.

Oligosaccharides and polysaccharides of the present invention, based on mannose, that have been found to increase cellularity and collagen deposition in cutaneous tissue include 1,4-beta-D-mannose oligosaccharides and polysaccharides, the latter being commonly referred to as a 1,4-beta-D-mannans. These materials include the 1,4-beta-D-mannose polymer isolated from the seeds of Phoenix canariensis and the oligosaccharides isolated therefrom (Villarroya and Petek, 1976). The beta-D-1,4 mannose oligosaccharides of from 4 to 12 mannose units and mixtures thereof are effective in causing an increase in cellularity and collagen deposition in areas of dermal tissue to which they have been applied.

Other mannose based oligosaccharides and polysaccharides including the phosphorylated oligosaccharides and phosphorylated polysaccharides isolated from the culture medium of the yeast Pichia holstii (Parolis, et al., 1996; Bretthauer et al., 1973), have been discovered to be effective in causing an increase in the cellularity and deposition of collagen in cutaneous tissue to which they are applied. The mannan isolated from Saccharomyces cerevisiae (Lee and Ballou, 1965) has also been found to be effective in this regard.

As a result of investigations described herein, it has now been discovered that certain sugars, notably, among others, maltopentaose; maltohexaose; maltoheptaose; 1,4-beta-D-mannopentaose; 1,4-beta-D-mannohexaose; the phosphorylated mannan obtained from Pichia holstii; the phosphorylated polysaccharide core (PPME) and pentasaccharide (PM5) obtained by acid hydrolysis of the mannan from Pichia holstii; and the mannan obtained from Saccharomyces cerevisiae, are effective tissue remodelling agents and effective anti-wrinkle compounds. Dermal application of these agents causes increases in fibroblast migration and collagen deposition and augmentation of skin thickness at the site of application in mammalian skin.

The present invention relates to the use of specific oligosaccharides and polysaccharides as anti-wrinkle and tissue remodelling agents. In this aspect, there is provided a method of ant-wrinkle and tissue remodelling treatment of an animal or human patient which comprises administration to the patient an effective amount of at least one oligosaccharide or polysaccharide or modification thereof.

In another aspect, this invention relates to the use of at least one phosphosugar-containing oligosaccharide or polysaccharide or derivative thereof in the preparation or manufacture of a cosmetic, pharmaceutical or veterinary composition for anti-wrinkle and tissue remodelling treatment. In this aspect, there is provided a cosmetic, pharmaceutical or veterinary composition which comprises at least one phosphorylated oligosaccharide or polysaccharide or derivative thereof, together with an acceptable cosmetic, pharmaceutical or veterinary carrier or diluent thereof.

Oligosaccharides or polysaccharides which may be used in accordance with the present invention comprise both naturally occurring and synthetic compounds including those containing or comprising phosphosugar residues, that is, sugar residues bearing at least one phosphate moiety. Particularly useful oligosaccharides and polysaccharides include those containing phosphomannoses, while useful oligosaccharides or polysaccharides include polysaccharides comprised of mannose and phosphomannose residues, maltohexaose, maltopentaose, yeast mannan. Presently some preferred agents include, but not limited to, maltopentaose, maltohexaose, maltoheptaose, 1,4-beta-D-mannopentaose, 1,4-beta-D-mannohexaose, the phosphorylated mannan obtained from Pichia holstii, the phosphorylated polysaccharide core (PPME) and pentasaccharide (PM5) obtained by acid hydrolysis of the mannan from Pichia holstii, the mannan obtained from Saccharomyces cerevisiae, amylose and amylopectin.

Suitable means to prepare PM5 can be found in WO 90/01938, incorporated herein by reference.

Whilst it is not intended that the present invention should be restricted in any way by a theoretical explanation of the mode of action of the carbohydrates in accordance with the invention, it is presently believed that these active compounds may exert their anti-wrinkle and tissue remodelling effect by attracting fibroblasts to the site of application and by inducing collagen deposition at this site. Accordingly, the active oligosaccharides, phospho-oligosaccharides, polysaccharides, phospho-polysaccharides or derivatives thereof may include any such compounds which are effective at causing fibroblast migration and increasing collagen production at the site of their administration.

The active anti-wrinkle and tissue remodelling agents in accordance with the present invention may be used to remodel and treat soft tissue defects including shallow or deep wrinkles of skin of the face and neck. These active agents may be used alone, in combination with one another or in combination with other carbohydrates, or in combination with other known tissue remodelling agents including collagen and hyaluronic acid.

Augmentation or remodelling refers to changing the structure of the dermis. This occurs due to production of new collagen by cells in the dermis stimulated by the oligosaccharide or polysaccharide according to the present invention. A remodelled or augmented dermis will give rise to skin which is less wrinkled, smoother in texture, firmer, plumper and more elastic.

The present invention may be carried out by application of topical creams containing oligosaccharides or polysaccharides capable of attracting fibroblasts and causing the production of collagen to the site of application. Injectable treatments typically commence with a course of one or more treatments over a period of a few months with maintenance treatments performed less frequently.

As a result of treatment according to the present invention, it is desirable for the skin to be less wrinkled, smoother in texture, firmer, plumper and more elastic.

There are no known other agents which, when applied topically can both attract fibroblasts and stimulate production collagen. The present invention provides the ability to produce endogenous collagen at desired sites in the skin. In contrast, other modes of treatment often require the addition of exogenous collagen to provide augmentation or remodelling of skin. This form of treatment has the serious disadvantage of the using non-human animal derived collagen, particularly bovine collagen which can be contaminated with infectious or deleterious agents such as viruses or prions.

Topical formulations typically include 0.1% to saturation of oligosaccharide or polysaccharide in a suitable carrier vehicle. Such vehicles are well known in the art and include encapsulation of the oligosaccharide or polysaccharide in liposomes or other forms of micro-encapsulation or microfine (about 2 μm to 20 μm) particles of oligosaccharide or polysaccharide undissolved in an anhydrous vehicle as described in European patent 0 572 494 (1999 Taylor).

Many drugs or compositions are given as pro-drugs to increase absorption. One form in this regard is to provide chemical modification to increase lipophilicity. Pro-drugs can be modified to the active drug by the body by specific or non-specific methods. Furthermore, non-specific conversion of pro-drug to drug can be by hydrolysis. Specific conversion of pro-drug to drug can occur by enzymes. Esterase enzymes in the skin will be capable of cleaving palmitate moieties attached to the sugars to increase lipophilicity.

Examples of methods suitable for use in modifying oligosaccharide or polysaccharide compounds suitable for the present invention can be found in the following journal articles: Raku T, Kitagawa M, Shimakawa H, Tokiwa Y. Enzymatic synthesis of trehalose esters having lipophilicity. J Biotechnol Feb. 13, 2003; 100(3):203-208; Redmann I, Pina M, Guyot B, Blaise P, Farines M, Graille J. Chemoenzymatic synthesis of glucose fatty esters. Carbohydr Res May 12,1997; 300(2):103-108; Tsuzuki W, Kitamura Y, Suzuki T, Kobayashi S. Synthesis of sugar fatty acid esters by modified lipase. Biotechnol Bioeng Aug. 5, 1999; 64(3):267-271; and Bousquet M P, Willemot R M, Monsan P, Boures E. Enzymatic synthesis of unsaturated fatty acid glucoside esters for dermo-cosmetic applications. Biotechnol Bioeng Jun. 20, 1999; 63(6):730-736. It will be appreciated, however, that other chemical modifications would also be suitable, depending on the type of modification required and the oligosaccharide or polysaccharide compound to be modified.

Injectable formulations would comprise the oligosaccharide or polysaccharide in solution of water or physiological saline. Suitable formulations would also include combination of the oligosaccharide or polysaccharide with other materials used for soft tissue augmentation, such as collagen or crosslinked hyaluronic add. The latter would provide the benefit of immediate soft tissue augmentation provided by the collagen or crosslinked hyaluronic acid with the longer term effects of the oligosaccharide or polysaccharide.

Fibroblasts in rats and humans are morphologically and functionally identical. Accordingly, results in a rat skin model can be extrapolated directly for human situations. The experimental results obtained by the present inventor clearly demonstrate the potential of the present invention in improving skin characteristics.

The present invention provides a clear and unexpected advance in the science of skin augmentation as there are no known other agents which have a demonstrated ability to both attract fibroblasts and to stimulate production of collagen to such an extent in skin.

The pharmaceutical forms suitable for injectable use include sterile aqueous solutions (where water soluble) or dispersions and sterile powders for the extemporaneous preparation of sterile injectable solutions or dispersion. The form should be sterile and must be fluid to the extent that easy syringability exists. It must be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as licithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It can be advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired effect in association with the required pharmaceutical carrier or diluent. The specification for the novel dosage unit forms of the invention can be dictated by and directly dependent on (a) any unique characteristics of the active material and the particular effect to be achieved, and (b) any limitations inherent in the art of compounding such an active material for the treatment of disease in living subjects having a diseased condition in which bodily health is impaired as herein disclosed in detail.

For topical applications, suitable diluents and cream bases are well known to the art and would be applicable for use in the present invention.

### EXAMPLES

### Example 1

The effects of intradermally injected glucose, maltose and maltopentaose on cell migration into the site of administration in the skin of normal rats. Healthy, specific pathogen free female Fischer rats 10-12 weeks of age (n=3) were anesthetised with ether and hair on their backs was clipped (electric miniclippers) from two areas of approximately 1.5 cm×4 cm either side of and paralleling the spinal column. The test and control substances were injected into the centre of 1 square cm defined areas. Substances were administered in such a fashion that each was injected into 4 different positions at least once in order to control for any possible anatomical positioning effect. Agents were dissolved in normal saline at a concentration of 100 mg/ml and sterile filtered prior to administration. Fifty microlitre injection volumes were given intradermally using 30 gauge needles and control injections consisted of 50 microlitres of sterile normal saline. Animals were sacrificed 48 hours following injection and injection sites were subjected to histological sectioning followed by microscopic examination. In this experiment, neither saline, glucose nor maltose had any effect on cellularity at the site of injection. The sections treated with maltopentaose showed a significant increase in cellularity (P=0.001) versus saline in the lower dermal layers.

### Example 2

The effects of intradermally injected dextran and maltopentaose on cell migration into the site of administration in the skin of normal rats. In a similar experiment to that outlined in Example 1 above, female Fischer rats, 10-12 weeks of age (n=3) were treated with 50 microlitre volumes of i) 50 mg/ml of clinical grade dextran (MW 71,400); ii) 50 mg/ml maltopentaose; or iii) normal saline using a 30 gauge needle. Skin injection sites were submitted for histological sectioning and examined microscopically 48 hours following injection. In this case, increased cellularity was noticed in the dextran injection sites but this was not statistically significantly different from the saline injected sites. The maltopentaose injected sites showed a clear and significant difference in cellularity versus saline.

### Example 3

The effects of intradermally injected maltotriose and maltopentaose on cell migration into the site of administration in the skin of normal rats. In a similar experiment to that outlined in Example 1 above, female Fischer rats, 10-12 weeks of age (n=2) were treated with 50 microlitre volumes of i) 100 mg/ml of maltotriose; ii) 100 mg/ml maltopentaose; or iii) normal saline using a 30 gauge needle. Skin injection sites were submitted for histological sectioning and examined microscopically 48 hours following injection. In this case, no increase in cellularity was noticed in the saline or maltotriose injection sites but as in Examples 1 and 2 above, the maltopentaose injected sites showed a clear and significant difference in cellularity versus saline.

### Example 4

The effects of intradermally injected maltopentaose and 1,4-beta-mannohexaose on cell migration into the site of administration in the skin of normal rats. In a similar experiment to that outlined in Example 1 above, female Fischer rats, 10-12 weeks of age (n=2) were treated with 50 microlitre volumes of i) 100 mg/ml of maltopentaose; ii) 100 mg/ml of 1,4-beta-mannohexaose; or iii) normal saline using a 30 gauge needle. Skin injection sites were submitted for histological sectioning and examined microscopically 48 hours following injection. In this case, increases in cellularity were observed in the maltopentaose and 1,4-beta-mannohexaose injection sites both of which were significantly different from the saline injection sites. Despite an apparently greater effect observed in the 1,4-beta-mannohexaose injection sites versus those for maltopentaose, the results were not significantly different.

### Example 5

The effects of intradermally injected amylose and the O-phosphorylated mannan isolated from Pichia holstii on cell migration into the site of administration in the skin of normal rats. In a similar experiment to that outlined in Example 1 above, female Fischer rats, 10-12 weeks of age (n=2) were treated with 50 microlitre volumes of i) 50 mg/ml of amylose; ii) 50 mg/ml of the O-phosphorylated mannan from Pichia holstii; or iii) normal saline using a 30 gauge needle. Skin injection sites were submitted for histological sectioning and examined microscopically 48 hours following injection. In this case, increases in cellularity were observed in the amylose and O-phosphorylated mannan injection sites both of which were significantly different from the saline injection sites.

### Example 6

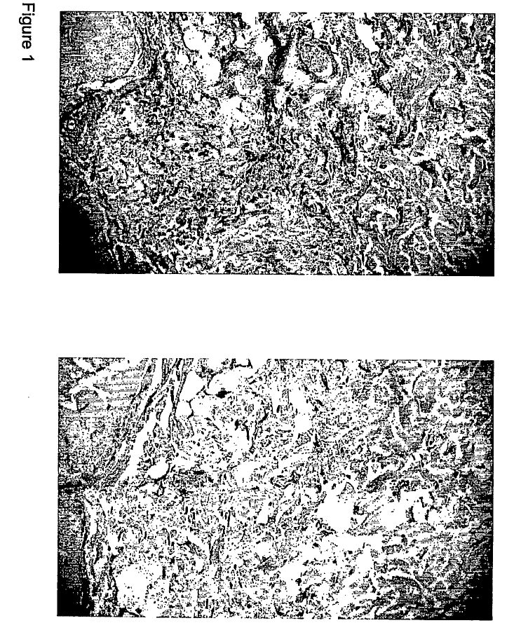

In this example, the effects of O-phosphorylated mannan on cellularity in skin following intradermal administration was examined. The methods used for this experiment were similar to those used in the Example 1. Following ether anaesthesia, hair was clipped from both sides of the abdomen of 8-10 week old female Fischer rats. Saline (50 microlitre) was injected into the centre of a defined 1 cm² region of skin, and O-phosphorylated mannan (50 microlitre; 50 mg/ml saline) was similarly injected on the contralateral side of the animals using a 30 gauge needle. Forty eight hours, 1, 2 and 4 weeks later, the animals were euthanased by CO₂ overdose and skin samples taken for histological assessment. Increased dermal cellularity (as described above) was dearly delineated in the areas that were treated after 48 hours, 1 and 2 weeks. FIG. 1 shows histological sections from saline and O-phosphorylated mannan treated skin sections taken 1 week after injection. After 1 month, an increase in cellularity remained evident, although reduced compared with the earlier time points.

### Example 7

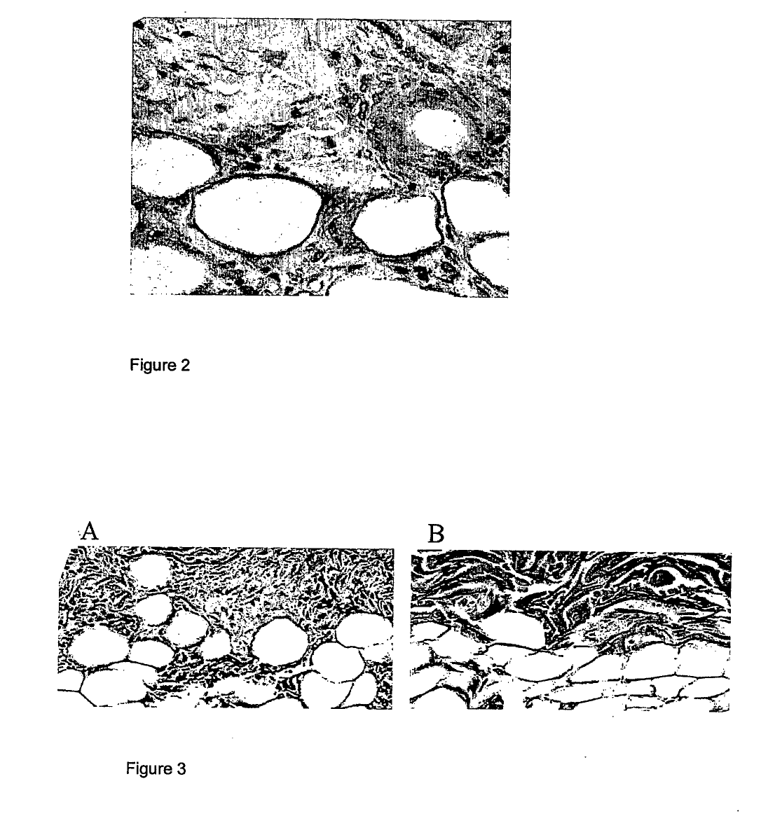

In the following example, pentasaccharide (PM5) and the maltose polysaccharide amylopectin were assessed for their ability to induce an increase in cellularity following injection into a localised area of skin. Following ether anaesthesia, hair was clipped from skin on both sides of the abdominal region of 8-10 week old female Wistar rats. Intradermal injections (via a 30 gauge needle) of either sterile saline (50 microliters), PM5 (50 microliters, 50 mg/ml in sterile saline) or amylopectin (50 microliters, 50 mg/ml in sterile saline) were placed in the centre of 1 cm² defined areas of abdominal skin. Forty-eight hours and 7 days after injection, groups of animals were euthanased by CO₂ overdose and skin samples were examined following the preparation of 6 μm histological sections that were stained with H&E and Gomori's (collagen) stain. In addition, immunohistochemistry using a mouse anti-rat prolyl 4-hydroxylase monoclonal antibody (Chemicon International Inc) in paraffin embedded skin sections was used to identify fibroblasts within the dermis. PM5-treated skin sections showed an increase in fibroblast-like cells, with the majority of the increase, and collagen deposited in the lower dermal layers. Amylopectin caused a marked “fibroblast-like” cellular infiltrate in both the upper and lower regions of the dermis (FIG. 2) that was associated with an increase in dermal collagen (as indicated Gomori's staining) (FIG. 3), and fibroblast numbers (as indicated by immunohistochemical staining).

The experimental data using rats clearly shows that selected 1,4 linked D-glucose oligosaccharides or polysaccharides have the unexpected and useful characteristic of being able to cause in vivo accumulation of fibroblasts in the skin and induce production of collagen. The accumulation of fibroblasts production of collagen allows skin, over time, to be less wrinkled, smoother in texture, firmer, plumper and more elastic.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Klein, A W, Wexler, P, Carruthers, A and Carruthers J, 1997. Treatment of facial furrows and rhytides. Dermatology Clinics, vol 15, 595-607.

Gilchrest, B A and Chiu, N. 1995. In: Merck Manual of Geriatrics. Section 15 Dermatologic and Sensory Organ Disorders Chapter 122 “Aging and the Skin” 1995 Merck & Co., Inc. Whitehouse Station N.J., USA.

Fenske, N A and Lober C W., 1986. Structural and functional changes of normal aging skin. Journal of the American Academy of Dermatology, vol 4, 571-585.

Contet-Audonneau, J L, Jeanmarie, C and Pauly, G, 1999. A histological study of human wrinkle structures: comparison between sun-exposed areas of the face, with or without wrinkles, and sun-protected areas. British Journal of Dermatology, vol 140, 1038-1047.

Gilchrest, B A, 1997. Treatment of photodamage with topical tretinoin: an overview. Journal of the American Academy of Dermatology, vol 36, s27-s36.

Moy, L S, Howe, K et al., 1996. Glycolic acid modulation of collagen production in human skin fibroblast cultures in vitro. Dermatological Surgery, vol 22, 439-441.

Kang, S., Fisher, G J et al., 1997. Photoaging and topical tretinoin: therapy, pathogenesis and prevention. Archives of Dermatology, vol 133, 1280-1284.

Villarroya H and Petek, F, 1976. Purification and properties of a beta-mannanase from alfalfa seeds. Biochimica er Biophysica Acta, vol438, 200-211.

Parolis, L A S, Duus, JØ, Parolis, H, Meldal, M and Bock, K. The extracellular polysaccharide of Pichia (Hansenula) holstii NRRL Y-2448: The structure of the phosphomannan backbone. Carbohydrate Research, vol 293, 101-117.

Bretthauer, R K, Kaczorowski, G J and Weise, M J, 1973. Characterization of a phosphorylated pentasaccharide isolated from Hansenula holstii NRRL Y-2448 phosphomannan. Biochemistry, vol 12, 1251-1256.

Lee, Y-C and Ballou, C E, 1965. Preparation of mannobiose, mannotriose and a new mannotetraose from Saccharomyces cerevisiae mannan. Biochemsitry, vol 4, 257-264.

1-17. (canceled)

18. A process for skin augmentation of a subject, the process comprising delivering to the skin of a subject an active component comprising a 1,4 linked D-glucose oligosaccharide or polysaccharide selected from the group consisting of 1,4 beta-manohexaose, O-phosphorylated mannan, purified D-mannose high molecular weight acid-resistant polysaccharide fragment (polysaccharide core fraction) of the exocellular phosphomannan produced by Pichia holstii, mixtures or combinations thereof, physically modified analogues thereof, and chemically modified analogues thereof wherein after delivery the oligosacchande or polysaccharide causes an accumulation of fibroblasts in the skin at or near to the site of delivery and induces production of collagen in the skin.

19. The process according to claim 18, wherein the oligosaccharide or polysaccharide is 1,4 beta-manohexaose.

20. The process according to claim 18, wherein the oligosaccharide or polysaccharide is O-phosphorylated mannan.

21. The process according to claim 18, wherein the oligosaccharide or polysaccharide is purified D-mannose high molecular weight acid-resistant polysaccharide fragment (polysaccharide core fraction) of the exocellular phosphomannan produced by Pichia holstii.

22. The process according to claim 22, wherein the purified D-mannose high molecular weight acid-resistant polysaccharide fragment comprises 6-O-phospho-alpha-D-mannose-(1,3)-alpha-D-mannose-(1,3)-alpha-D-mannose-(1,3)-alpha-D-mannose-(1,2)-alpha-D-mannose.

23. The process according to claim 18, wherein the oligosaccharide or polysaccharide is injected directly into the skin or applied topically to the skin.

24. The process according to claim 18, wherein the oligosaccharide or polysaccharide is delivered to the skin at a concentration of 0.001 to 100% by weight.

25. The process according to claim 24, wherein the oligosaccharide or polysaccharide is delivered to the skin at a concentration of 0.01 to 70% by weight.

26. The process according to claim 25, wherein the oligosaccharide or polysaccharide is delivered at a concentration of 0.1 to 30% by weight.

27. The process according to claim 26, wherein the oligosaccharide or polysaccharide is delivered at a concentration of 1 to 20% by weight.

28. The process according to claim 18, resulting in the treated skin being less wrinkled, smoother in texture, firmer, plumper and more elastic compared with corresponding untreated skin.

29. A cosmetic preparation for skin augmentation of a subject comprising an effective amount of a 1,4 linked D-glucose oligosaccharide or polysaccharide selected from the group consisting of 1,4 beta-manohexaose O-phosphorylated mannan purified D-mannose high molecular weight acid-resistant polysaccharide fragment (polysaccharide core fraction) of the exocellular phosphomannan produced by Pichia holstii mixtures or combinations thereof physically modified analogues thereof and chemically modified analogues thereof in a suitable diluent or excipient, the oligosaccharide or polysaccharide being capable of causing an accumulation of fibroblasts in the skin and inducing production of collagen in the skin

30. The cosmetic preparation according to claim 29, wherein the oligosaccharide or polysaccharide is 1,4 beta-manohexaose.

31. The cosmetic preparation according to claim 29, wherein the oligosaccharide or polysaccharide is O-phosphorylated mannan.

32. The cosmetic preparation according to claim 29, wherein the oligosaccharide or polysaccharide is purified D-mannose high molecular weight acid-resistant polysaccharide fragment (polysaccharide core fraction) of the exocellular phosphomannan produced by Pichia holstii.

33. The cosmetic preparation according to claim 29, wherein the purified D-mannose high molecular weight acid-resistant polysaccharide fragment comprises 6-O-phospho-alpha-D-mannose-(1,3)-alpha-D-mannose-(1,3)-alpha-D-mannose-(1,3)-alpha-D-mannose-(1,2)-alpha-D-mannose.
